(19) United States
(12) Patent Application Publication
(10) Pub. No.: US 2002/0068096 A1
(43) Pub. Date: Jun. 6, 2002

(54) Antimonocytic activity of betel leaf extracts

(76) Inventors: Santu Bandyopadhyay, Calcutta (IN); Bikash Pal, Calcutta (IN); Samir Bhattacharya, Calcutta (IN); Mitali Ray, Calcutta (IN); Keshab Chandra Roy, Calcutta (IN)

Correspondence Address: FITZPATRICK CELLA HARPER & SCINTO, 30 ROCKEFELLER PLAZA, NEW YORK, NY, 10112, US

(21) Appl. No.: 09/772,003

(22) Filed: Jan. 30, 2001

(30) Foreign Application Priority Data
Dec. 4, 2000 (IN) PCT/IN00/00118

(51) Int. Cl. A61K 35/78

(52) U.S. Cl. 424/725

(57) ABSTRACT

This invention relates to anti-monocytic activity of betel leaf extracts and this anti monocytic activity of betel leaf extracts suggest its use to treat myeloid leukemia in animal and human beings.

### FIELD OF INVENTION

This invention relates to anti-Monocytic activity of betel leaf extracts. Myeloid leukemia is lethal and usually does not respond to chemotherapy leading to poor prognosis. Anti Monocytic activity of betel leaf extracts suggest its potential use to treat myeloid leukemia. This invention also relates to a method of treating Myeloid leukemia using the betel leaf extract to an animal including human beings suffering from Myeloid leukemia.

### BACKGROUND AND PRIOR ART REFERENCES

Betel leaves have a strong pungent aromatic flavor and are widely used as a masticatory. Generally, mature or over mature leaves, which have ceased growing but not yet become brittle are used for chewing. The basic preparation for chewing purposes consists of betel leaf smeared with hydrated lime and catechu to which scrapings of arecanut are added; flavorings such as coconut shavings, clove, cardamom, fennel, powdered liquorice, nutmeg and also tobacco are used according to one's taste. In some places prepared pan is covered with silver or gold leaf. As a masticatory, it is credited with many properties: it is aromatic, digestive, stimulant and carminative. Medicinally it is useful in catarrhal and pulmonary infections; it is also used for poultices. The effects of chewing of betel with arecanut and other adjuncts are the excitation of the salivary glands and the irritation of the mucous membrane of the mouth. The red coloration produced is due to a pigment in the arecanut, which manifests itself under the action of alkali in time and catechu. A mild degree of stimulation is produced, resulting in a sensation of warmth and well being, besides imparting a pleasant odor. The most important factor determining the aromatic value of the leaf is the amount and particularly the nature of the essential oil present. Betel leaves from different regions vary in smell and taste. The most pungent is the Sanchi type, while the most mild and sweet ones are from Madras. The betel leaves contain essential oils, the content of oil varies from 0.7 to 2.6 percent depending upon the varieties of leaves. The oil consists of phenols and terpens. The higher the proportion of phenol oil, the better the quality. An isomer of eugenol named chavibetol (betel phenol; 4-allyl-2-hydroxy-1-methoxy benzene) is considered to be the characteristic constituent of betel oil. It is however, absent in Indian samples. Betel oil of Indian types contain as a predominant phenolic constituent. Oil of betel has been used in the treatment of various respiratory catarrhs, under as a local application either by gargle or by inhalation in diphtheria. It has carminative properties. It exhibits in different action on the central nervous system of mammals; lethal doses produce deep narcosis leading to death with a few hours. The essential oil and extracts of the leaves possess activity against several Gram-positive and Gram-negative bacteria such as Micrococcus pyogenes var. albus, Bacillus subtilis and B. megaterium, Diplococcus pneumoniae, Streptococcus pyogenes, Escherichia coli, Salmonella typhosa, Vibrio comma, Shigella dysenteriae, Proteus vulgaris, Pseudomonas solanacaerum, Sarcina lutea and Erwinia carotorora. The essential oil and leaf extracts also showed antifungal activity against Asperigillus niger and A. oryzae, Curvularia lunata and Fusarium oxysporum. The oil is found to be lethal in about 5 minutes to the protozoa Paramaeceum caudatum (Wealth of India, Vol. 8, pg. 84-94). It inhibits the growth of Vibrio cholerae, Salmonella typhosum and Shigella flexneri and Escherichia coli. Steam—distillate of the leaves showed activity against Mycobacterium tuberculosis.

Myeloid leukemia is usually subdivided into two groups: Acute Myeloid Leukemia (AML) and Chronic Myeloid Leukemia (CML). AML is characterized by an increase in the number of myeloid cells in the marrow and an arrest in their maturation, frequently resulting in hematopoietic insufficiency. In the United States, the annual incidence of AML is approximately 2.4 per 100,000 and it increases progressively with age to a peak of 12.6 per 100,000 adults 65 years of age or older. Despite improved therapeutic approaches, prognosis of AML is very poor around the globe. Even in the United States, five-year survival rate among patients who are less than 65 years of age is less than 40%.

During the last decade this value was 15. Similarly, the prognosis of CML is also very poor in spite of advancement of clinical medicine.

### OBJECTS OF THE INVENTION

The main object of the invention is for treating myeloid leukemia in animals including human beings using betel leaf extract.

Another object is to provide a composition comprising betel leaf extract useful for the treatment of myeloid leukemia.

### SUMMARY OF THE INVENTION

To meet the above objects, the invention provides anti momocytic activity of betel leaf extract and this activity is employed for treating myeloid leukemia in animals including human beings.

### DETAILED DESCRIPTION OF THE INVENTION

Accordingly, the present invention provides a new use of betel leaf extract namely anti monocytic activity. This anti Monocytic activity of betel leaf extracts is used for treating myeloid leukemia in animals including human beings.

In an embodiment, a pharmaceutical composition useful for the treatment of myeloid leukemia, said composition comprising effective amount of betel leaf extract together with or associated with a pharmaceutically acceptable additive.

In still another embodiment of the invention, the additive is selected in such a manner it does not interfere with the activity of betel leaf extract.

Yet, another embodiment of the invention, the additive is selected from nutrients such as proteins, carbohydrates and sugar, talc, magnesium sterate, cellulose, calcium carbonate, starch-gelatin paste and/or pharmaceutically acceptable carriers, excipient, diluent or solvent.

Yet another embodiment of the invention, the betel leaf extract or the composition is administered orally or intramuscularly.

Still another embodiment of the invention, the oral route is in the form of capsule, syrup, concentrate, powder or granules.

Yet another embodiment of the invention, the ratio of betel leaf extract to the additive is in the range between 10 to 1.

Yet another embodiment of the invention, the betel leaf extract or the composition is administered at a dosage level between 5 to 20 mg/kg of body weight for alternate days for one month.

Yet another embodiment of the invention, the betel leaf extract or the composition reduces the monocytes content by 80%.

Yet another embodiment of the invention, the betel leaf extract or the composition is used for the treatment of myeloid leukemia.

Yet another embodiments of the invention, the betel leaf extract is administered together with or associated with a pharmaceutically acceptable additive.

Yet another embodiment of the invention, the additive is selected in such a manner it does not interfere with the activity of betel leaf extract.

Yet, another embodiment of the invention, the additive is selected from nutrients such as proteins, carbohydrates and sugar, talc, magnesium sterate, cellulose, calcium carbonate, starch-gelatin paste and/or pharmaceutically acceptable carriers.

Yet another embodiment of the invention, the betel leaf extract or the composition is administered orally or intramuscularly.

Still another embodiment of the invention, the oral route is in the form of capsule, syrup, concentrate, powder or granules.

Yet another embodiment of the invention, the ratio of betel leaf extract to the additive is in the range between 10 to 1.

Yet another embodiment of the invention, the betel leaf extract or the composition is administered at a dosage level between 5 to 20 mg/kg of body weight for alternate days for one month.

Yet another embodiment of the invention, the betel leaf extract or the composition reduces the viability of monocytes by 80%.

Yet another embodiment of the invention, the betel leaf extract is obtained by crushing the betel leaf or extracting the crushed leafs with water or organic solvents such as alcohol, carbontetrachloride, chloroform and acetone.

One more embodiment of the present invention provides the preparation of betel leaf extracts comprising the following steps:

1) washing of the fresh leaves of Piper betel and homogenizing in a mixture blender;

2) sonicating in an ultrasonic bath with 2 to 3 bursts each for 15 minutes and filtering the extract, if desired repeating the extraction at least once and drying; and

3) lyophilizing the extract to get a semi-solid mass

Yet another embodiment of the invention, the betel leaf (Piper betle) is selected from the following types namely Wild type, Climber type, Bangla type and Sweet type.

### BRIEF DESCRIPTION OF THE DRAWINGS

FIG. 1: represents destruction of monocytes from human PBMC after incubation with betel leaf extract.

The following examples are given by way of explanation and for illustration only and these examples should not be construed in any manner to limit the scope of the invention.

### EXAMPLES

### Example 1

34.14 gm of fresh leaves of Piper betle thoroughly washed in sterile water was homogenized with 100 ml of glass distilled water in a mixture-blender. It was then sonicated in an ultrasonic bath with 3 burst each for 15 min. The extract was filtered through Whatman No.1 filter paper and the filtrate was collected. This process of extraction was repeated three times. The combined extract was lyophilized yielding a semi-solid mass weighing 1.17 gm. This was then tested for biological activity.

### Example 2

The fresh leaves of Piper betle weighing 21.68 gm homogenized with distilled water (60 ml) in a mixture—blender and then sonicated in an ultrasonic bath with 2 burst each for 15 min. It was allowed to be extracted overnight or 16 hours. Filtering through Whatman No.1 filter paper separated the material extracted in water. This type of treatment for extraction was repeated for three times. The combined extract was evaporated to dryness in a flash evaporator under reduced pressure at 45° C. The residual substance was then dried in a desiccator under high vacuum and the semi-solid mass weighing 0.59 gm was tested for biological activity.

Properties of the extract material

The biologically active material obtained by examples 1 and 2 has the following properties:

1) The dried semisolid prepared as stated above was a dark colored material soluble in water and dimethyl sulfoxide.

2) Thin layer chromatography of the active material shows five spots having R_{f} 0.75, 0.64, 0.50, 0.40 and 0.33 in the solvent system of n-butanol, acetic acid and water in the ratio of 9:5:7 respectively.

3) The HPLC analysis of the active material using Intersil ODS-3 (4.6×250 mm) analytical column, solvent system methanol and water in the ratio of 4:1 and a flow rate of 1.0 ml/min., detection at 217 nm resolved the material into eleven peaks with the retention time of 2.69, 4.27, 5.95, 6.97, 7.49, 9.39, 11.20, 12.40, 15.53, 18.90 and 21.49 mins.

### Example 3

Preparation of human peripheral blood mononuclear cells (PBMC):

Heparinized whole blood (collected from normal individuals) was subjected to Ficoll Hypaque density gradient centrifugation. Cells in the interface were washed twice with phosphate buffered saline (PBS) and then re-suspended in medium RPMI-1640 supplemented with 10%. Fetal Bovine Serum.

Incubation of hPBMC with betel leaf extract:

PBMC (5.0×10⁶ cells) were cultured overnight (18 hours) at 37° C. in 5% CO₂ in a total volume of 2.0 ml RPMI+10% FBS in 24 well plates in the presence or absence of betel leaf extracts (12.5-mg/ml final concentration). At the end of the incubation period, PBMC were washed twice with PBS and used for flow cytometry for the detection of Monocyte destruction.

Monitoring of Light Scattering induced by lymphocytes and monocytes by Flow Cytometry:

hPBMC were incubated with betel extracts, washed with PBS once and resuspended in PBS containing 1% paraformaldehyde. Cells were analyzed in a flow cytometer (FACS Calibur, Becton Dickinson)

Results:

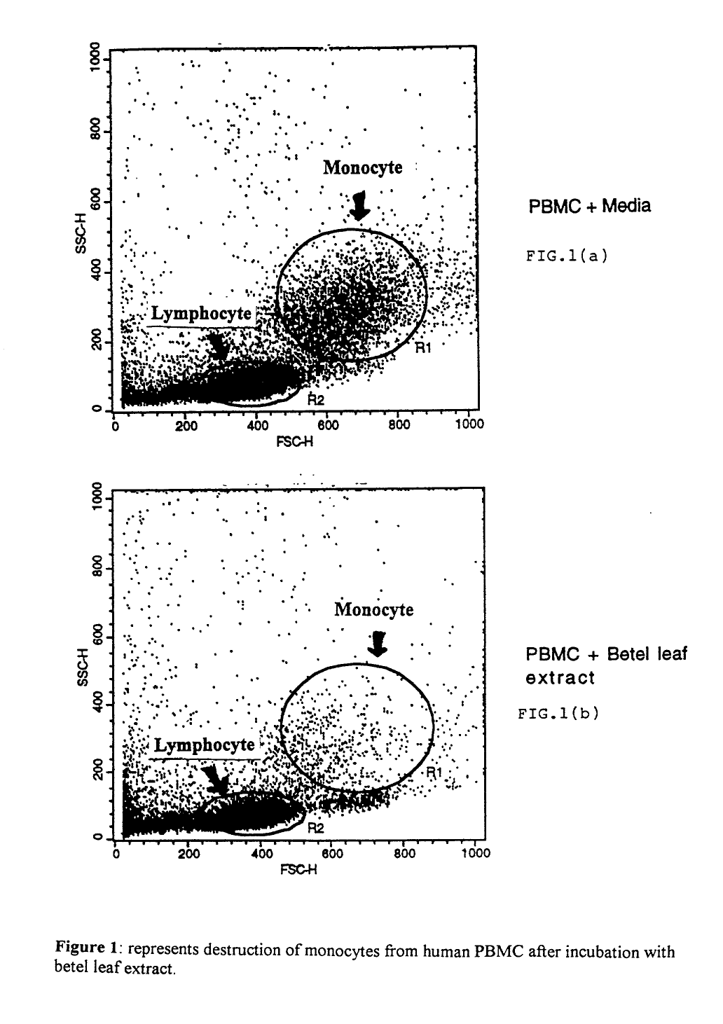

As shown in FIG. 1A, peripheral blood mononuclear cells had expected proportion of monocytes (R1) and lymphocytes (R2). In contrast hPBMC incubated overnight with betel leaf extract (wild type) had unaffected lymphocytes (FIG. 1B, R2), but had almost complete disappearance of monocytes (FIG. 1B, R1). It appears that the betel leaf reduces viability of monocytes by at least 80%.

Discussion:

Thus, our results suggest that anti-Monocytic property of betel leaf extract could be exploited for treatment of myeloid leukemia. The effective amount required for treating human beings is believed to be 5 to 20 mg/kg of body weight for alternate days; with a preferred amount being 8 to 20 mg/kg of body weight.

1. A method of treating Myeloid leukemia in animals including human beings using the betel leaf extract, lyophilized extract or a composition comprising betel leaf extract, said method comprising administering a pharmaceutically effective amount of betel leaf extract, lyophilized extract or a composition comprising betel leaf extract to an animal including human beings suffering from Myeloid leukemia.

2. A method as claimed in claim 1 wherein, the composition comprising betel leaf extract associated with or in combination with a pharmaceutically acceptable additive.

3. A method as claimed in claim 1 wherein, the additive is selected in such a manner that does not interfere with the activity of betel leaf extract.

4. A method as claimed in claim 1 wherein, the additive is selected from nutrients such as proteins, carbohydrates, sugar, talc, magnesium sterate, cellulose, calcium carbonate, starch-gelatin paste and/or pharmaceutically acceptable carriers, excipient, diluent or solvent.

5. A method as claimed in claim 1 wherein, the betel leaf extract or the composition is administered orally or intramuscularly.

6. A method as claimed in claim 1 wherein, the oral route is in the form of capsule, syrup, concentrate, powder or granules.

7. A method as claimed in claim 1 wherein, the ratio of betel leaf extract to the additive is in the range between 1-10:10-1.

8. A method as claimed in claim 1 wherein, the betel leaf extract is obtained by crushing the betel leaf or extracting the crushed leafs with water or organic solvents such as alcohol, carbontetrachloride, chloroform and acetone.

9. A method as claimed in claim 1 wherein, the betel leaf extract or the composition administered at a dosage level between 5 to 20 mg/kg of body weight for alternate days for one month.

10. A method as claimed in claim 1 wherein, the composition reduces the viability of monocytes by 80%.

11. A pharmaceutical composition useful for the treatment of myeloid leukemia, said composition comprising effective amount of betel leaf extract or lyophilized extract together with or associated with a pharmaceutically acceptable additive.

12. A composition as claimed in claim 11 wherein, the additive is selected in such a manner it does not interfere with the activity of betel leaf extract.

13. A composition as claimed in claim 11 wherein, the additive is selected from nutrients such as proteins, carbohydrates, sugar, talc, magnesium sterate, cellulose, calcium carbonate, starch-gelatin paste and/or pharmaceutically acceptable carriers.

14. A composition as claimed in claim 11 wherein, the composition is administered orally or intramuscularly.

15. A composition as claimed in claim 11 wherein, the oral route is in the form of capsule, syrup, concentrate, powder or granules.

16. A composition as claimed in claim 11 wherein, the ratio of betel leaf extract to the additive is in the range between 1-10:10-1.

17. A composition as claimed in claim 11 wherein, the composition is administered at a dosage level between 5 to 20 mg/kg of body weight for alternate days for one month.

18. A composition as claimed in claim 11 wherein, the betel leaf extract or the composition reduces the monocytes content by 80%.

19. A composition as claimed in claim 11 wherein, the betel leaf extract used is having the following properties:
i) The dried sample is a dark colored material soluble in water and dimethyl sulfoxide,
ii) Thin layer chromatography of the active material shows five spots having R_{f} 0.75, 0.64, 0.50, 0.40 and 0.33 in the solvent system of n-butanol, acetic acid and water in the ratio of 9:5:7 respectively, and
iii) The HPLC analysis of the active material using Intersil ODS-3 (4.6×250 mm) analytical column, solvent system methanol and water in the ratio of 4:1 and a flow rate of 1.0 ml/min., detection at 217 nm resolved the material into eleven peaks with the retention time of 2.69, 4.27, 5.95, 6.97, 7.49, 9.39, 11.20, 12.40, 15.53, 18.90 and 21.49 min.

20. Use of betel leaf extract for the treatment of myeloid leukemia in animals including human beings.

21. Use as claimed in claim 20 wherein, the betel leaf extract is administered together with or associated with a pharmaceutically acceptable additive.

22. Use as claimed in claim 20 wherein, the additive is selected in such a manner it does not interfere with the activity of betel leaf extract.

23. Use as claimed in claim 20 wherein, the additive is selected from nutrients such as proteins, carbohydrates and sugar, talc, magnesium sterate, cellulose, calcium carbonate, starch-gelatin paste and/or pharmaceutically acceptable carriers.

24. Use as claimed in claim 20 wherein, the betel leaf extract or the composition is administered orally or intramuscularly.

25. Use as claimed in claim 20 wherein, the oral route is in the form of capsule, syrup, concentrate, powder or granules.

26. Use as claimed in claim 20 wherein, the ratio of betel leaf extract to the additive is in the range between 1-10:10-1.

27. Use as claimed in claim 20 wherein, the betel leaf extract or the composition is administered at a dosage level between 5 to 20 mg/kg of body weight for alternate days for one month.

28. Use as claimed in claim 20 wherein, the betel leaf extract or the composition reduces the viability of monocytes by 80%.

29. Use of betel leaf as a composition comprising betel leaf extract to provide anti-monocytic activity in blood cells.

30. A method of destroying monocytes present in human peripheral mononuclear cells (PBMSs) using the betel leaf extract, lyophilized extract or a composition comprising betel leaf extract, said method comprising treating PBMCs with a pharmaceutically effective amount of betel leaf extract, lyophilized extract or a composition comprising betel leaf extract.

31. A method as claimed in claim 1 wherein, the composition comprising betel leaf extract associated with or in combination with a pharmaceutically acceptable additive.

32. A method as claimed in claim 1 wherein, the additive is selected in such a manner that does not interfere with the activity of betel leaf extract.

33. A method as claimed in claim 1 wherein, the additive is selected from nutrients such as proteins, carbohydrates, sugar, talc, magnesium sterate, cellulose, calcium carbonate, starch-gelatin paste and/or pharmaceutically acceptable carriers, excipient, diluent or solvent.

34. A method as claimed in claim 1 wherein, the betel leaf extract or the composition is administered orally or intramuscularly.

35. A method as claimed in claim 1 wherein, the oral route is in the form of capsule, syrup, concentrate, powder or granules.

36. A method as claimed in claim 1 wherein, the ratio of betel leaf extract to the additive is in the range between 1-10:10-1.

37. A method as claimed in claim 1 wherein, the betel leaf extract is obtained by crushing the betel leaf or extracting the crushed leafs with water or organic solvents such as alcohol, carbon tetrachloride, chloroform and acetone.

38. A method as claimed in claim 1 wherein, the betel leaf extract or the composition administered at a dosage level between 5 to 20 mg/kg of body weight for alternate days for one month.

39. A method as claimed in claim 1 wherein, the composition reduces the viability of monocytes at least by 80%.
